(19) United States
(12) Patent Application Publication
(10) Pub. No.: US 2026/0363895 A1
(43) Pub. Date: Nov. 19, 2026

(54) BIMAXILLARY SPLINT

(71) Applicant: MEDSTAR HEALTH, INC., Columbia, MD (US)

(72) Inventor: Stephen B. Baker, Columbia, MD (US)

(21) Appl. No.: 19/671,663

(22) Filed: May 8, 2026

Related U.S. Application Data
(60) Provisional application No. 63/806,167, filed on May 15, 2025.

(51) Int. Cl. A61F 5/058

(52) U.S. Cl. CPC A61F 5/05891

(57) ABSTRACT

A bimaxillary splint for fixing each of the maxilla and mandible of a patient in a selected position includes a substantially U-shaped splint body having opposing upper and lower body portions. The upper body portion has a plurality of maxillary receptacles for receiving associated maxillary dental structures of the patient. The lower body portion has a plurality of mandibular receptacles for receiving associated mandibular dental structures of the patient. A plurality of maxillary retention features is configured to engage the maxillary dental structures and/or interproximal spaces between adjacent maxillary dental structures to form an interference fit engagement between the upper body portion and the patient’s maxillary dental structures. A plurality of mandibular retention features is configured to engage the mandibular dental structures and/or interproximal spaces between adjacent mandibular dental structures to form an interference fit engagement between the lower body portion and the patient’s mandibular dental structures.

### RELATED APPLICATION

This application claims priority from U.S. Provisional Application No. 63/806,167, filed 15 May 2025, the subject matter of which is incorporated herein by reference in its entirety.

### TECHNICAL FIELD

This disclosure generally relates to a bimaxillary splint, and, in particular, to a bimaxillary splint for fixing each of the maxilla and mandible of a patient in a selected position. This disclosure also generally relates to a bimaxillary fixation system for use in a bimaxillary surgery, and a method for performing a bimaxillary surgery using said bimaxillary fixation system.

### Background

Certain procedures, such as, but not limited to, mandibular and maxillary fracture repairs and osteotomies, certain head and neck tumor reconstruction surgeries, and/or orthognathic surgeries, often involve bringing the one or both of the mandible (i.e., lower jaw) and maxilla (i.e., upper jaw) into a target position and at least temporarily fixing one or both of the mandible and maxilla in the target position via intermaxillary fixation. For example, FIGS. 1-3 depict a patient 100 in various stages of a double jaw (e.g., bimaxillary) surgery. In FIG. 1, the patient’s maxilla 102 and mandible 104 are each in a pre-op position as neither the maxilla 102 nor the mandible 104 has been manipulated into a target position. FIG. 2 depicts the patient’s maxilla 102 after having been manipulated (e.g., cut and/or repositioned) into a target position. At least the patient’s maxilla 102 is then temporarily fixed/maintained in this target position using one or more fixation devices while the maxilla 102 is secured in the target positioned using hardware (e.g., plates and/or screws). Often, the patient’s mandible 104 is also temporarily fixed in position (e.g., the pre-op position) via one or more fixation devices while the patient’s maxilla 102 is being secured. Once the patient’s maxilla 102 is fixed/secured in the target position via the hardware, the patient’s mandible 104 may be manipulated to a target position/alignment, temporarily fixed/maintained in the target position, and fixed/secured in the target position in the same or a similar manner as the maxilla 102. FIG. 3 depicts the patient’s mandible 104 after having been manipulated into its target position.

Arch bars are often used in the above-mentioned procedures to achieve intermaxillary fixation. Certain known arch bars are configured to be secured to the teeth of the patient via wire loops or direct dental cement bonding. Securing these dental arch bars and/or maintaining these dental arch bars secured to the patient is often difficult, especially if the patient has pre-existing orthodontic appliances and/or missing teeth. As an alternative to these dental arch bars, other known arch bars are at least temporarily secured to the patient via the direct placement of screws into the patient’s maxilla and/or mandible. While these screwed-in arch bars overcome some of the issues of the dental arch bars, they are not without their own drawbacks. For example, these screwed-in arch bars often have to be removed in a secondary/follow-up procedure, thereby increasing the time and cost expenditures of both the patient and the medical facility performing the procedures. As another example of a disadvantage, the screwed-in arch bars may be associated with possible dental injury due to screw placement.

### Summary

In an aspect, alone or in combination with any other aspect, a bimaxillary splint for fixing each of the maxilla and mandible of a patient in a selected position is provided. The bimaxillary splint comprises a substantially U-shaped splint body having opposing upper and lower body portions. The upper body portion has a plurality of maxillary receptacles for receiving associated maxillary dental structures of the patient. The lower body portion has a plurality of mandibular receptacles for receiving associated mandibular dental structures of the patient. A plurality of maxillary retention features is on the upper body portion in and/or adjacent to the maxillary receptacles. The maxillary retention features are configured to engage the maxillary dental structures and/or interproximal spaces between adjacent maxillary dental structures to form an interference fit engagement between the upper body portion and the patient’s maxillary dental structures. A plurality of mandibular retention features is on the lower body portion in and/or adjacent to the mandibular receptacles. The mandibular retention features are configured to engage the mandibular dental structures and/or interproximal spaces between adjacent mandibular dental structures to form an interference fit engagement between the lower body portion and the patient’s mandibular dental structures.

In an aspect, alone or in combination with any other aspect, a bimaxillary fixation system for use in a bimaxillary surgery is provided. The bimaxillary fixation system comprises a plurality of the above bimaxillary splint. The plurality of bimaxillary splints comprises first and second bimaxillary splints. The first bimaxillary splint is configured such that one of the maxillary and mandibular receptacles are in an associated pre-op position while the other of the maxillary and mandibular receptacles are in an associated target position. The second bimaxillary splint is configured such that the maxillary and mandibular receptacles are in their associated target positions.

In an aspect, alone or in combination with any other aspect, a method for performing a bimaxillary surgery is provided. The method comprises providing the above bimaxillary fixation system. Through use of the first bimaxillary splint, a first one of the maxilla and mandible of the patient is retained in an associated pre-op position while a second one of the maxilla and mandible is manipulated into an associated target position from an associated pre-op position. With the second one of the maxilla and mandible maintained in its associated target position via the first bimaxillary splint, the second one of the maxilla and mandible is fixed in its associated target position. After fixing the second one of the maxilla and mandible in its associated target position, the first bimaxillary splint is removed. Through use of the second bimaxillary splint, the second one of the maxilla and mandible is retained in its associated target position while the first one of the maxilla and mandible is manipulated into an associated target position from its associated pre-op position. With the maxilla and mandible maintained in their associated target positions via the second bimaxillary splint, the first one of the maxilla and mandible is fixed in its associated target position. After fixing the first one of the maxilla and mandible in its associated target position, the second bimaxillary splint is removed.

### BRIEF DESCRIPTION OF THE DRAWINGS

For a better understanding, reference may be made to the accompanying drawings, in which:

FIGS. 1-3 illustrate various stages of a patient undergoing a bimaxillary surgery;

FIG. 4 is a perspective top view of a first example bimaxillary splint according to one aspect of the present invention;

FIG. 5 is a perspective bottom view of the aspect of FIG. 4;

FIG. 6 is a top view of the aspect of FIG. 4;

FIG. 7 is a bottom view of the aspect of FIG. 4;

FIG. 8 is a flowchart of a first example method for performing a bimaxillary surgery;

FIG. 9 is a perspective top view of a second example bimaxillary splint according to one aspect of the present invention;

FIG. 10 is a perspective bottom view of the aspect of FIG. 9;

FIG. 11 is a top view of the aspect of FIG. 9;

FIG. 12 is a bottom view of the aspect of FIG. 9; and

FIG. 13 is a flowchart of a second example method for performing a bimaxillary surgery.

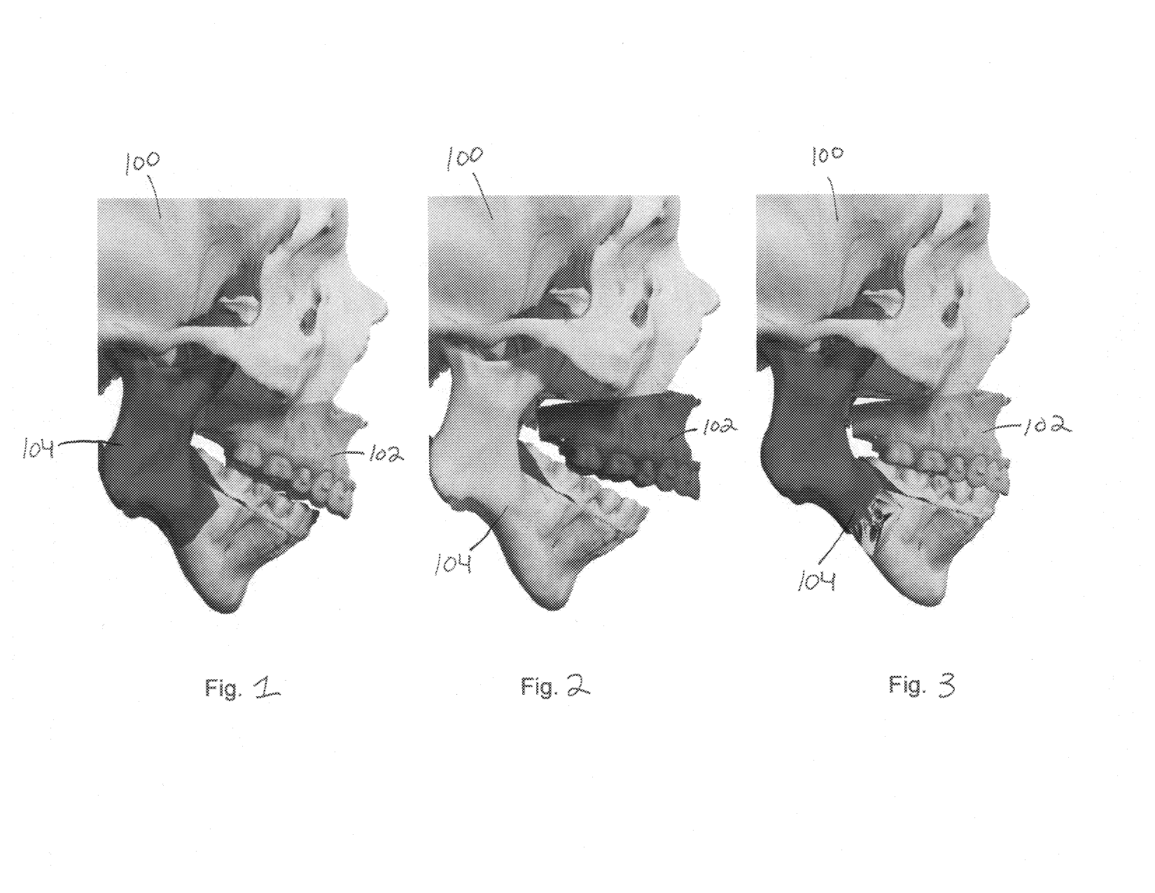

### DETAILED DESCRIPTION

Unless defined otherwise, all technical and scientific terms used herein have the same meaning as is commonly understood by one of ordinary skill in the art to which the present disclosure pertains.

As used herein, the term “patient” can refer to any warm-blooded organism including, but not limited to, human beings, pigs, rats, mice, cats, dogs, goats, sheep, horses, monkeys, apes, rabbits, cattle, farm animals, livestock, etc.

As used herein, the term “medical professional” can be used interchangeably to refer to an individual who prepares for, assists with, and/or performs a surgical procedure and/or provides care to a patient. A medical professional can be, for example, a doctor, a dentist, a physician' s assistant, a student, a nurse, a caregiver, or the like.

As used herein, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” can be interpreted to include X and Y.

As used herein, the phrase “at least one of X and Y” can be interpreted to include X, Y, or a combination of X and Y. For example, if an element is described as having at least one of X and Y, the element may, at a particular time, include X, Y, or a combination of X and Y, the selection of which could vary from time to time. In contrast, the phrase “at least one of X” can be interpreted to include one or more Xs.

It will be understood that when an element is referred to as being “on,” etc., another element, it can be directly on the other element or intervening elements may also be present. It will also be appreciated by those of skill in the art that references to a structure or feature that is disposed “adjacent” another feature may not have portions that overlap or underlie the adjacent feature.

Spatially relative terms, such as “lower,” “upper” and the like, may be used herein for ease of description to describe one element or feature’s relationship to another element(s) or feature(s) as illustrated in the Figures. It will be understood that the spatially relative terms can encompass different orientations of a device in use or operation, in addition to the orientation depicted in the Figures. For example, if a device in the Figures is inverted, elements described as “lower” would then be oriented as “upper” elements.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or Figures unless specifically indicated otherwise.

As used herein, a “dental structure” of a patient can include a natural tooth, a natural tooth modified with one or more dental prosthetics (e.g., a crown or veneer) and/or orthodontic appliances (e.g., brackets), a prosthetic tooth (e.g., a tooth of a denture), a dental implant and/or prosthetic at least partially anchored to and/or implanted in a bone (e.g., a maxilla and/or mandible) of a patient (e.g., a post that is surgically inserted into a patient’s maxilla or mandible to provide a foundation for at least one prosthetic tooth and/or a denture), a dental bridge, an alveola (the bone into which the teeth are located), and the like. Therefore, a patient having multiple dental structures may have one or more types of dental structures and/or any combination of dental structure types.

As used herein, a “maxillary dental structure” or “maxillary dental structures” refers/refer to a dental structure or dental structures that are positioned in or otherwise associated with a patient’s maxilla. Conversely, a “mandibular dental structure” or “mandibular dental structures” refers/refer to a dental structure or dental structures that are positioned in or otherwise associated with a patient’s mandible.

One of ordinary skill in the art will understand that the term “dental”, as used herein, could be interchangeable with the terms “alveolar” and/or “dentoalveolar”, as appropriate for a particular use environment. For example, a medical professional could choose to get retention on alveolar structures and possibly bypass embrasure retention as desired for a particular patient, using the teachings of the present disclosure

The invention comprises, consists of, or consists essentially of the following features, in any combination.

FIGS. 4-7 illustrate an example bimaxillary splint 400 for use in at least temporarily fixing each of a patient’s maxilla and mandible in a selected position and/or in a selected arrangement relative to one another. The bimaxillary splint 400 includes a splint body 402 having a base 404 configured to be located between maxillary and mandibular dental structures of the patient. As such, the base 404 and, accordingly, the splint body 402 are substantially U-shaped so as to conform to the maxillary and mandibular dental arches of the patient. The base 404 also defines opposing upper and lower body portions 406, 506 of the splint body 402. Therefore, the upper body portion 406 is on and includes an upper base portion side of the base 404, while the lower body portion 508 is on and includes an opposite lower base portion side of the base 404.

The upper base portion 408 includes one or more maxillary receptacles 410 (here a plurality of maxillary receptacles 410) for at least partially receiving associated maxillary dental structures of the patient. The maxillary receptacles 410 are at least partially defined by an inner lingual-side surface 412 of the upper base portion 408, an inner facial-side surface 414 of the upper base portion 408 that is opposite the lingual-side surface 412, and an inner end surface 416 of the upper base portion 408 that extends between the inner lingual-side surface 412 and the inner facial-side surface 414. Similarly, the lower base portion 508 includes one or more mandibular receptacles 510 (here a plurality of mandibular receptacles 510) for at least partially receiving associated mandibular dental structures of the patient. The mandibular receptacles 510 are also at least partially defined by an inner lingual-side surface 512 of the lower base portion 508, an inner facial-side surface 514 of the lower base portion 508 that is opposite the lingual-side surface 512, and an inner end surface 516 of the lower base portion 508 that extends between the inner lingual-side surface 512 and the inner facial-side surface 514. The number and/or geometric configuration of the maxillary and mandibular receptacles 410, 510 may be patient specific so that the bimaxillary splint 400 may correspond (e.g., directly correspond) to the number, geometric shape, and/or type of the patient’s maxillary and mandibular dental structures.

In addition to including the upper base portion 408, the upper body portion 406 may include maxillary retention features 418 for selectively retaining the maxillary dental structures in the maxillary receptacles 410 after they have been received therein. In other words, the maxillary retention features 418 are configured to selectively engage the maxillary dental structures and/or interproximal spaces between adjacent maxillary dental structures to form an interference fit engagement between the upper body portion 406 and the patient’s maxillary dental structures that selectively retains the maxillary dental structures in the maxillary receptacles 410 during use. An “interproximal embrasure” as defined herein is the opening/space located between two serially adjacent dental structures. The maxillary retention features 418 of the example configuration shown in FIGS. 4 and 6 comprise maxillary retention projections 420 that are in and/or adjacent to the maxillary receptacles 410. Each maxillary retention projection 420 may extend from at least one of the inner surfaces 412, 414, 416 of the upper base portion 408 and be located so as to be in line with and/or define a maxillary division 422 between two serially adjacent maxillary receptacles 410. Accordingly, the maxillary retention projections 420 are configured to extend into interproximal embrasures of the patient’s maxillary dental structures and at least partially form at least a portion of the interference fit engagement between the upper body portion 406 and the patient’s maxillary dental structures during use.

Interproximal embrasures often have several zones or portions. For example, an occlusal/incisal embrasure portion of an interproximal embrasure is typically located between the curved proximal surfaces of adjacent posterior/anterior dental structures, occlusal/incisal to the proximal contact. A facial embrasure portion of an interproximal embrasure is typically located between the curved proximal surfaces of adjacent dental structures, facial to the proximal contact. A lingual embrasure portion of an interproximal embrasure is typically located between the curved proximal surfaces of adjacent dental structures, lingual to the proximal contact. It should be noted that the term “lingual embrasure portion” and a “lingual direction” is utilized broadly so as to be applicable to both the maxilla and mandibular dental structures. As such, the lingual direction, as used herein, is opposite to a facial direction regardless of when referring to the maxillary dental structures or the mandibular dental structures. A gingival embrasure portion of an interproximal embrasure is typically located between curved proximal surfaces of adjacent dental structures, gingival to the proximal contact.

As shown in FIGS. 4 and 6, the maxillary retention projections 420 may each include an occlusal/incisal projection portion 424, a lingual projection portion 426, and/or a facial projection portion 428, though a geometric shape, size, location, and presence of these projection portions 424, 426, 428 in a maxillary retention projection 420 is dependent upon the particular geometry, arrangement, type, and/or number of the patient’s maxillary dental structures and/or interproximal embrasures between the patient’s maxillary dental structures. The occlusal/incisal projection portions 424 are each configured to extend vertically upward from the inner end surface 416 of the upper base portion 408 toward and/or into the occlusal/incisal embrasure portion of an associated interproximal embrasure. The lingual projection portions 426 are each configured to extend facially from the inner lingual-side surface 412 of the upper base portion 408 toward and/or into the lingual embrasure portion of an associated interproximal embrasure. Each facial projection portion 428 is configured to extend lingually from the inner facial-side surface 414 of the upper base portion 408 toward and/or into the facial embrasure portion of an associated interproximal embrasure.

In certain bimaxillary splint configurations, such as the one shown in FIGS. 4 and 6, at least one of the facial projection portions 428 and/or at least one of the lingual projection portions 426 may extend upward beyond the inner facial-side surface 414 and/or the inner lingual-side surface 412 onto a maxillary retention tab 430. In particular, the example splint body 402 shown in FIGS. 4 and 6 includes a plurality of maxillary retention tabs 430 that extend substantially vertically upward from the upper base portion 408, though the splint body 402 may be configured to have only one maxillary retention tab 430, only one maxillary retention tab 430 onto which a facial projection portion 428 extends, and/or only one maxillary retention tab 430 onto which a lingual projection portion 426 extends. The maxillary retention tabs 430 are each located such that at least a portion of each maxillary retention tab 430 is in line with an associated maxillary division 422, and at least one of the maxillary retention tabs 430 (here, a plurality or all of the maxillary retention tabs 430) are located on the facial side of the mandibular receptacles 410. Each facial-side maxillary retention tab 430 also includes an inner facial-side surface 432 onto which an associated facial projection portion 428 extends and from which at least a portion of the associated facial projection portion 428 extends in the lingual direction toward and/or into a facial embrasure portion of an associated interproximal embrasure. Increasing the vertical length of the facial projection portions 428 via use of the facial-side maxillary retention tabs 430 at least partially results in an increase in length of an interface and/or engagement between the facial projection portions 428 and their associated interproximal embrasures. This increase in length may also result in at least one facial projection portion 428 extending lingually into the gingival embrasure portion of an associated interproximal embrasure, further increasing the engagement between the at least one facial projection portion 428 and its associated interproximal embrasure.

As shown in at least FIGS. 4 and 6, because the maxillary retention tabs 430 are each in line with an associated maxillary division 422, the facial-side maxillary retention tabs 430 are spaced from one another along the substantially U-shaped extension of the base 404. This spacing produces an opening 434 between serially adjacent facial-side maxillary retention tabs 430. The openings 434 not only permit physical and visual access to the patient’s maxillary dental structures, they also allow the bimaxillary splint 400 to be useable with patients that have pre-existing orthodontic appliances (e.g., brackets) since the pre-existing orthodontic appliances can extend into the openings 434 without disrupting or significantly disrupting the engagement and/or fit between the bimaxillary splint 400 and the maxillary dental structures.

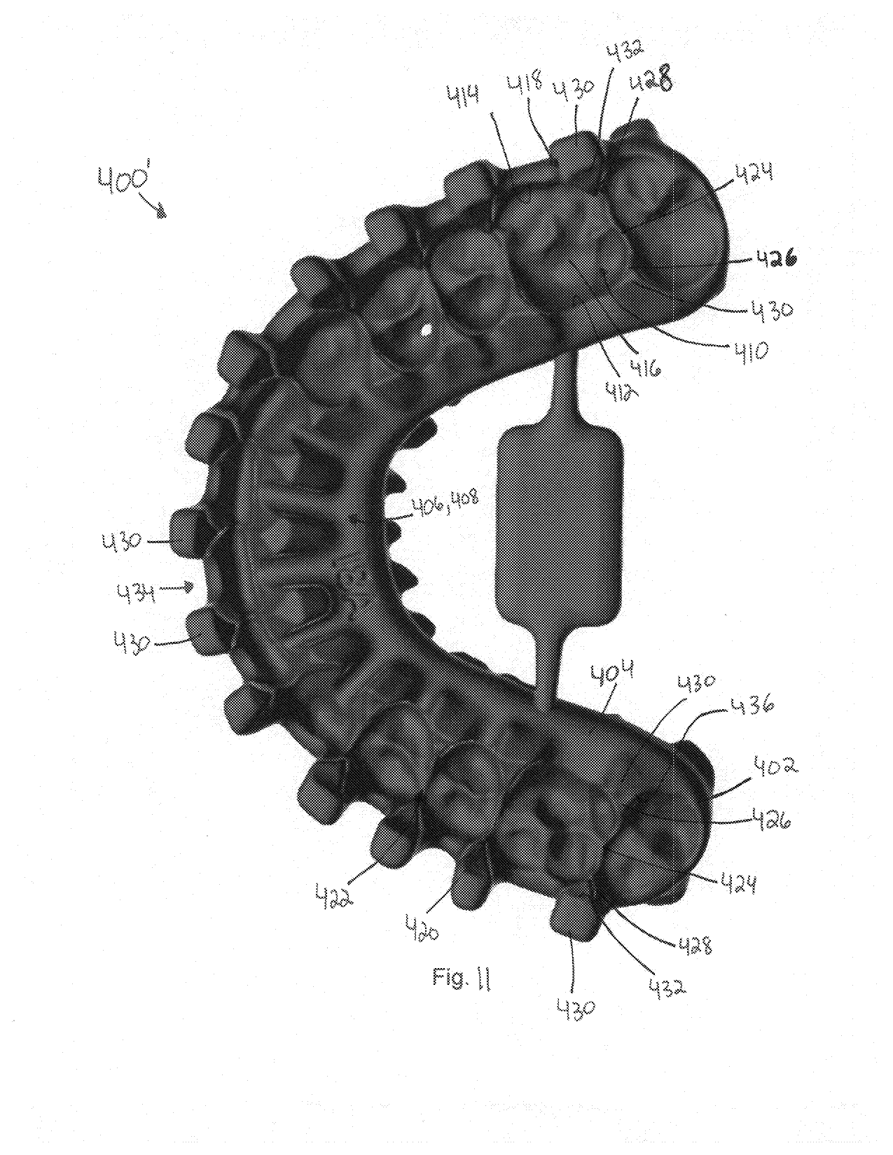

Although the maxillary retention tabs 430 are shown as being only or at least mostly on the facial side of the maxillary receptacles 410, the maxillary retention tabs 430 may also or instead be on a lingual side of the maxillary receptacles 410. In a bimaxillary splint configuration that includes a plurality of lingual-side maxillary retention tabs 430, the lingual-side maxillary retention tabs 430 may also be spaced from one another along the substantially U-shaped extension of the base 404 to produce the openings 434 between serially adjacent lingual-side maxillary retention tabs 430. However, unlike the facial-side maxillary retention tabs 430, each lingual-side maxillary retention tab 430 has an inner lingual-side surface (e.g., see 436 in FIG. 9) onto which an associated lingual projection portion 426 extends and from which at least a portion of the associated lingual projection portion 426 extends in the facial direction toward and/or into a lingual embrasure portion of an associated interproximal embrasure. Accordingly, a splint body 402 configuration that includes at least one lingual-side maxillary retention tab 430 may also include at least one lingual projection portion 426 that extends upward beyond the inner lingual-side surface 412 of the upper base portion 408 onto an associated lingual-side maxillary retention tab 430, thereby increasing the length of an interface and/or engagement between the at least one lingual projection portion 426 and its associated interproximal embrasure. Similarly to the above, this increase in length may result in at least one lingual projection portion 426 extending facially into the gingival embrasure portion of an associated interproximal embrasure, further increasing the engagement between the at least one lingual projection portion 426 and its associated interproximal embrasure.

While the maxillary retention tabs 430 are provided to at least partially increase the vertical length of the facial and/or lingual projection portions 428, 426, such length increases may also or instead be achieved via increasing the vertical height of the inner facial-side surface 414 and/or inner lingual-side surface 412 of the upper base portion 408. A desired facial and/or lingual projection portion 428, 426 height may thus still be achieved even in configurations of the splint body 402 that omit the maxillary retention tabs 430.

In addition to or instead of comprising maxillary retention projections 420, the maxillary retention features 418 may comprise the inner lingual-side surface 412 and the inner facial-side surface 414 of the upper base portion 408. In particular, a distance between the inner lingual-side surface 412 and the inner facial-side surface 414 at each maxillary receptacle 410 may be configured such that the inner lingual-side surface 412 and the inner facial-side surface 414 engage the maxillary dental structures on opposing sides of the maxillary dental structures (e.g., on lingual and facial sides of the maxillary dental structures) to such a degree that at least a portion of the interference fit engagement between the upper body portion 406 and the patient’s maxillary dental structures is formed.

Regardless of whether the maxillary retention features 418 of a bimaxillary splint 400 designed in accordance with the present teachings comprise the maxillary retention projections 420 and/or the inner lingual- and facial-side surfaces 412, 414, the maxillary retention features 418 engage the upper body portion 406 with the patient’s maxillary dental structures without the use of fasteners (e.g., metallic fasteners), wire loops, dental cement bonding, and/or other hardware and/or fastening mechanisms.

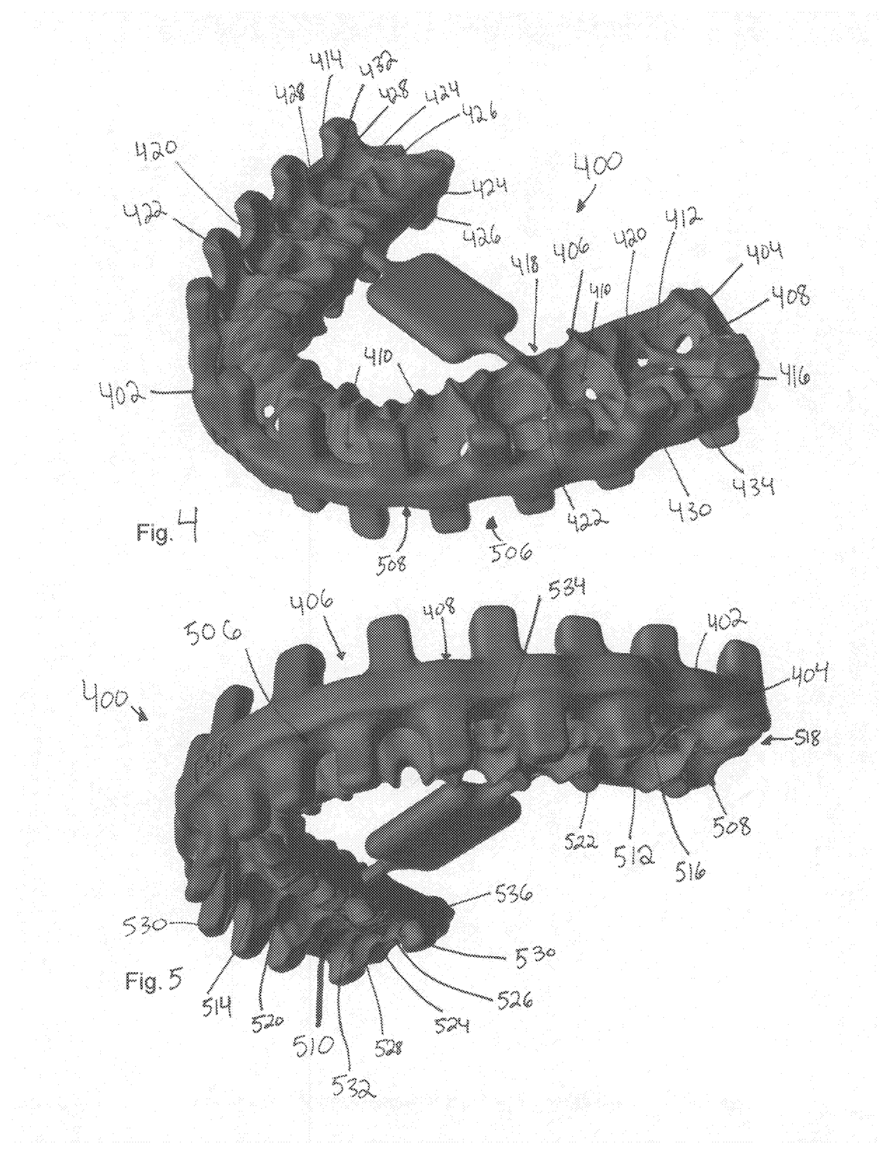

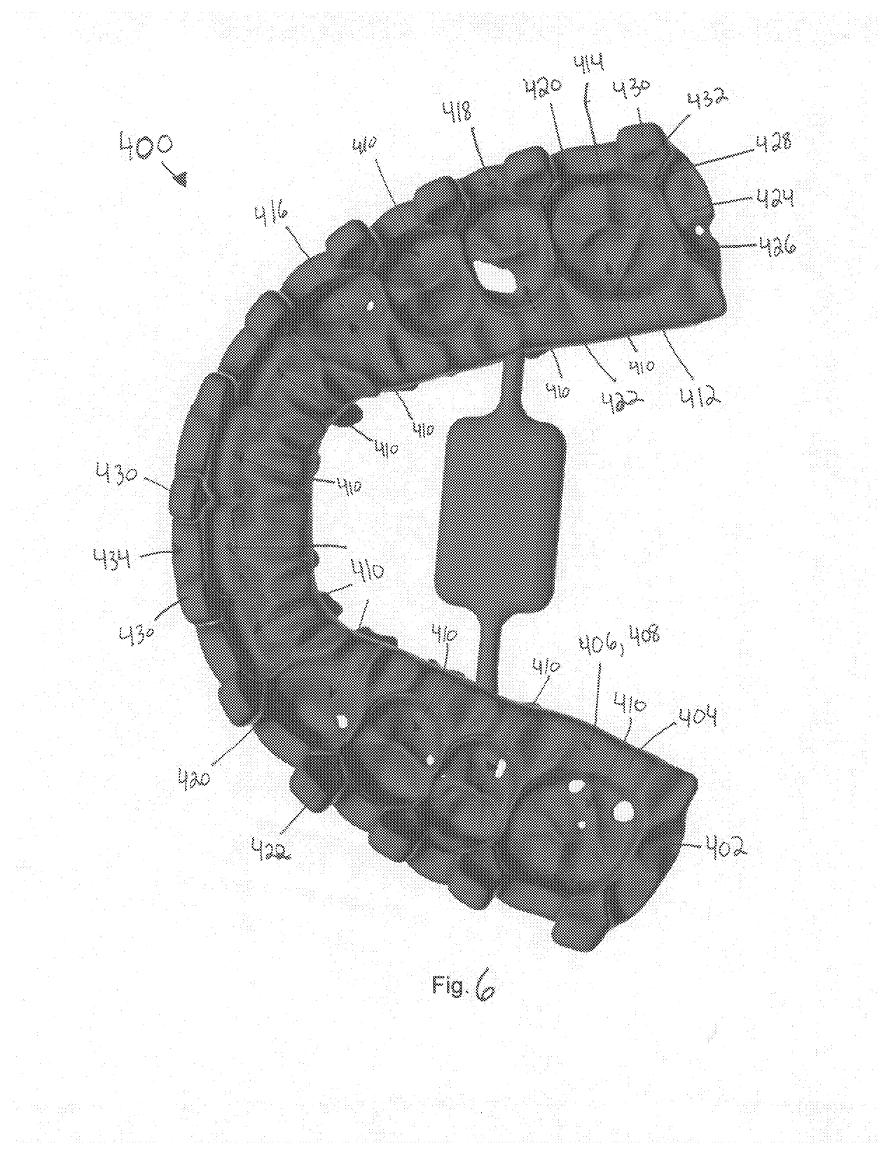

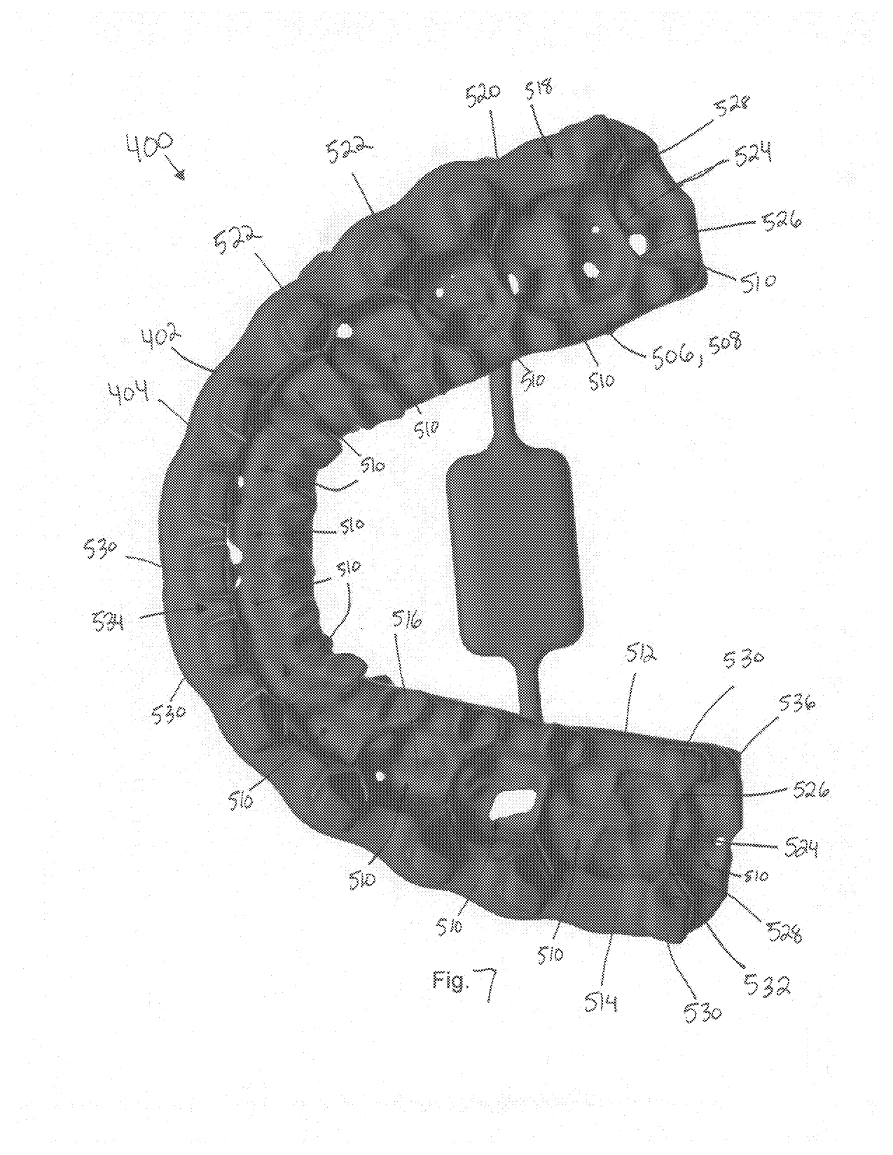

Returning to FIGS. 5 and 7, the lower body portion 506 may also include mandibular retention features 518 for selectively retaining the mandibular dental structures in the mandibular receptacles 510 after they have been received therein. In other words, the mandibular retention features 518 are configured to selectively engage the mandibular dental structures and/or interproximal spaces between adjacent mandibular dental structures to form an interference fit engagement between the lower body portion 506 and the patient’s mandibular dental structures that selectively retains the mandibular dental structures in the mandibular receptacles 510 during use. The mandibular retention features 518 of the example configuration shown in FIGS. 5 and 7 comprise mandibular retention projections 520 that are in and/or adjacent to the mandibular receptacles 510. Each mandibular retention projection 520 may extend from at least one of the inner surfaces 512, 514, 516 of the lower base portion 508 and be located so as to be in line with and/or define a mandibular division 522 between two serially adjacent mandibular receptacles 510. Accordingly, the mandibular retention projections 520 are configured to extend into interproximal embrasures of the patient’s mandibular dental structures and form at least a portion of the interference fit engagement between the lower body portion 506 and the patient’s mandibular dental structures during use.

The mandibular retention projections 520 may each include an occlusal/incisal projection portion 524, a lingual projection portion 526, and/or a facial projection portion 528, though a geometric shape, size, location, and presence of these projection portions in a mandibular retention projection 520 is dependent upon the particular geometry, arrangement, type, and/or number of the patient’s mandibular dental structures and/or interproximal embrasures between the patient’s mandibular dental structures. The occlusal/incisal projection portions 524 are each configured to extend vertically downward from the inner end surface 516 of the lower base portion 508 toward and/or into the occlusal/incisal embrasure portion of an associated interproximal embrasure. The lingual projection portions 526 are each configured to extend facially from the inner lingual-side surface 512 of the lower base portion toward 508 and/or into the lingual embrasure portion of an associated interproximal embrasure. Each facial projection portion 528 is configured to extend lingually from the inner facial-side surface 514 of the lower base portion 508 toward and/or into the facial embrasure portion of an associated interproximal embrasure.

In certain bimaxillary splint configurations, such as the one shown in FIGS. 5 and 7, at least one of the facial projection portions 528 and/or at least one of the lingual projection portions 526 may extend downward beyond the inner facial-side surface 514 and/or the inner lingual-side surface 512 onto a mandibular retention tab 530. In particular, the example splint body 402 shown in FIGS. 5 and 7 includes a plurality of mandibular retention tabs 530 that extend substantially vertically downward from the lower base portion 508, though the splint body 402 may be configured to have only one mandibular retention tab 530, only one mandibular retention tab 530 onto which a facial projection portion 528 extends, and/or only one mandibular retention tab 530 onto which a lingual projection portion 526 extends. The mandibular retention tabs 530 are each located such that at least a portion of each mandibular retention tab 530 is in line with an associated mandibular division 522, and at least one of the mandibular retention tabs 530 (here, a plurality of the mandibular retention tabs 530) are located on the facial side of the mandibular receptacles 510. Each facial-side mandibular retention tab 530 also includes an inner facial-side surface 532 onto which an associated facial projection portion 528 extends and from which at least a portion of the associated facial projection portion 528 extends in the lingual direction toward and/or into a facial embrasure portion of an associated interproximal embrasure. Increasing the vertical length of the facial projection portions 528 via use of the facial-side mandibular retention tabs 530 at least partially results in an increase in length of an interface and/or engagement between the facial projection portions 528 and their associated interproximal embrasures. This increase in length may also result in at least one facial projection portion 528 extending lingually into the gingival embrasure portion of an associated interproximal embrasure, further increasing the engagement between the at least one facial projection portion 528 and its associated interproximal embrasure.

As shown in at least FIGS. 5 and 7, because the mandibular retention tabs 530 are each in line with an associated mandibular division 522, the facial-side mandibular retention tabs 530 are spaced from one another along the substantially U-shaped extension of the base 404. This spacing produces an opening 534 between serially adjacent facial-side mandibular retention tabs 530. Similarly to that described above, the openings 534 not only permit physical and visual access to the patient’s mandibular dental structures, they also allow the bimaxillary splint 400 to be useable with patients that have pre-existing orthodontic appliances (e.g., brackets) since the pre-existing orthodontic appliances can extend into the openings 534 without disrupting or significantly disrupting the engagement and/or fit between the bimaxillary splint 400 and the mandibular dental structures.

Although the mandibular retention tabs 530 are shown as being predominately on the facial side of the mandibular receptacles 510, the mandibular retention tabs 530 may also or instead be on a lingual side of the mandibular receptacles 510. In a bimaxillary splint configuration that includes a plurality of lingual-side mandibular tabs 530, the lingual-side mandibular retention tabs 530 may also be spaced from one another along the substantially U-shaped extension of the base 404 to produce the openings 534 between serially adjacent lingual-side mandibular retention tabs 530. However, unlike the facial-side mandibular retention tabs 530, each lingual-side mandibular retention tab 530 has an inner lingual-side surface 536 onto which an associated lingual projection portion 526 extends and from which at least a portion of the associated lingual projection portion 526 extends in the facial direction toward and/or into a lingual embrasure portion of an associated interproximal embrasure. Accordingly, a splint body configuration that includes at least one lingual-side mandibular retention tab 530 (such as the splint body configuration shown in FIGS. 5 and 7) may also include at least one lingual projection portion 526 that extends downward beyond the inner lingual-side surface 512 of the lower base portion 508 onto an associated lingual-side mandibular retention tab 530, thereby increasing the length of an interface and/or engagement between the at least one lingual projection portion 526 and its associated interproximal embrasure. Similarly to the above, this increase in length may result in at least one lingual projection portion 526 extending facially into the gingival embrasure portion of an associated interproximal embrasure, further increasing the engagement between the at least one lingual projection portion 526 and its associated interproximal embrasure.

While the mandibular retention tabs 530 are provided to at least partially increase the vertical length of the facial and/or lingual projection portions 528, 526, such length increases may also or instead be achieved via increasing the vertical height of the inner facial-side surface 514 and/or inner lingual-side surface 512 of the lower base portion 508. A desired facial and/or lingual projection portion 528, 516 height may thus be achieved even in configurations of the splint body 402 that omit the mandibular retention tabs 530.

In addition to or instead of comprising mandibular retention projections 520, the mandibular retention features 518 may comprise the inner lingual-side surface 512 and the inner facial-side surface 514 of the lower base portion 508. In particular, a distance between the inner lingual-side surface 512 and the inner facial-side surface 514 at each mandibular receptacle 510 may be configured such that the inner lingual-side surface 512 and the inner facial-side surface 514 engage the mandibular dental structures on opposing sides of the mandibular dental structures (e.g., on lingual and facial sides of the mandibular dental structures) to such a degree that at least a portion of the interference fit engagement between the lower body portion 506 and the patient’s mandibular dental structures is formed.

Regardless of whether the mandibular retention features 518 of a bimaxillary splint 400 designed in accordance with the present teachings comprise the mandibular retention projections 520 and/or the inner lingual- and facial-side surfaces 512, 514, the mandibular retention features 518 engage the lower body portion 506 with the patient’s mandibular dental structures without the use of fasteners (e.g., metallic fasteners), wire loops, dental cement bonding, and/or other hardware and/or fastening mechanisms.

The base 404, maxillary retention projections 420 (when provided), the mandibular retention projections 520 (when provided), the maxillary retention tabs 430 (when provided), and/or the mandibular retention tabs 530 (when provided), may be integrally formed as a one-piece monolithic construction. However, at least one of the base 404, the maxillary retention projections 420 (when provided), the mandibular retention projections 520 (when provided), the maxillary retention tabs 430 (when provided), and/or the mandibular retention tabs 530 (when provided) may be formed separately from at least one other of the base 404, the maxillary retention projections 420 (when provided), the mandibular retention projections 520 (when provided), the maxillary retention tabs 430 (when provided), and/or the mandibular retention tabs 530 (when provided), with the separately formed element(s) being formed on the “other” element(s) and/or being subsequently connected to the “other” element(s).

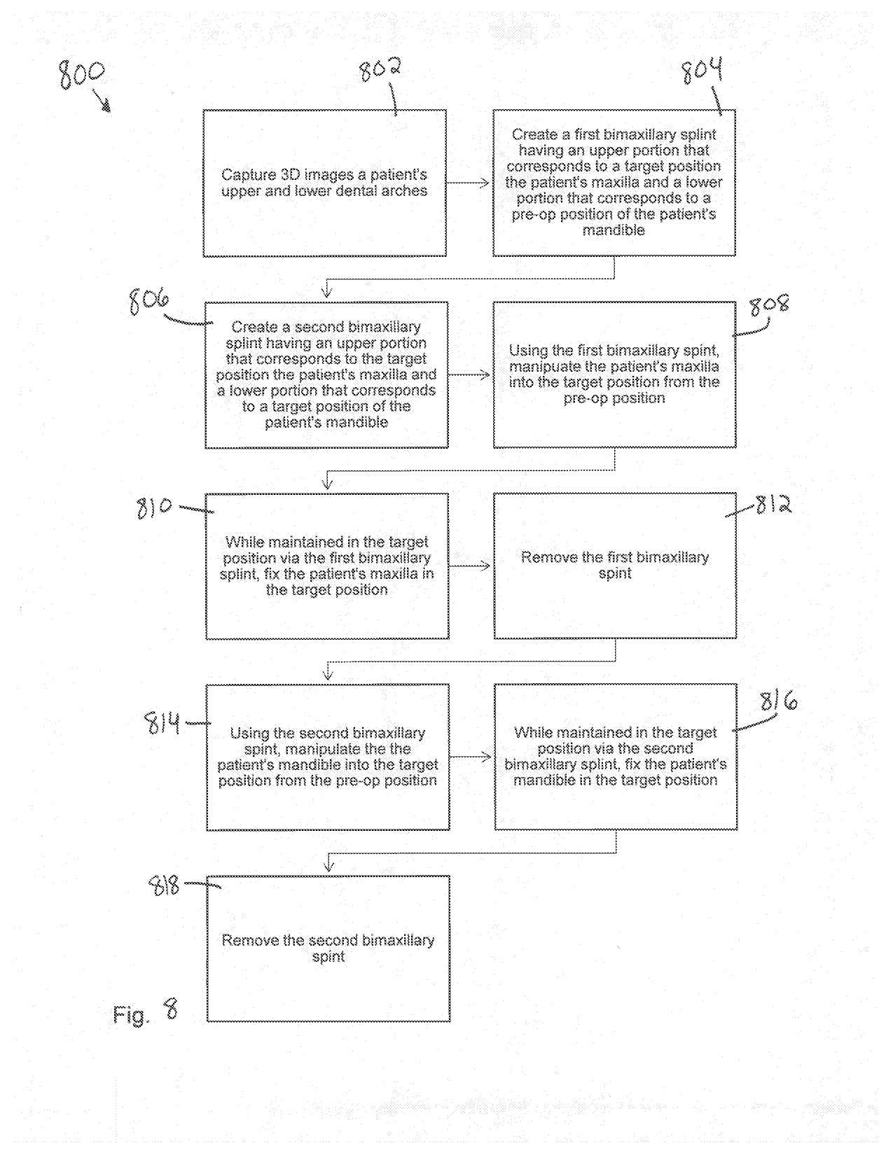

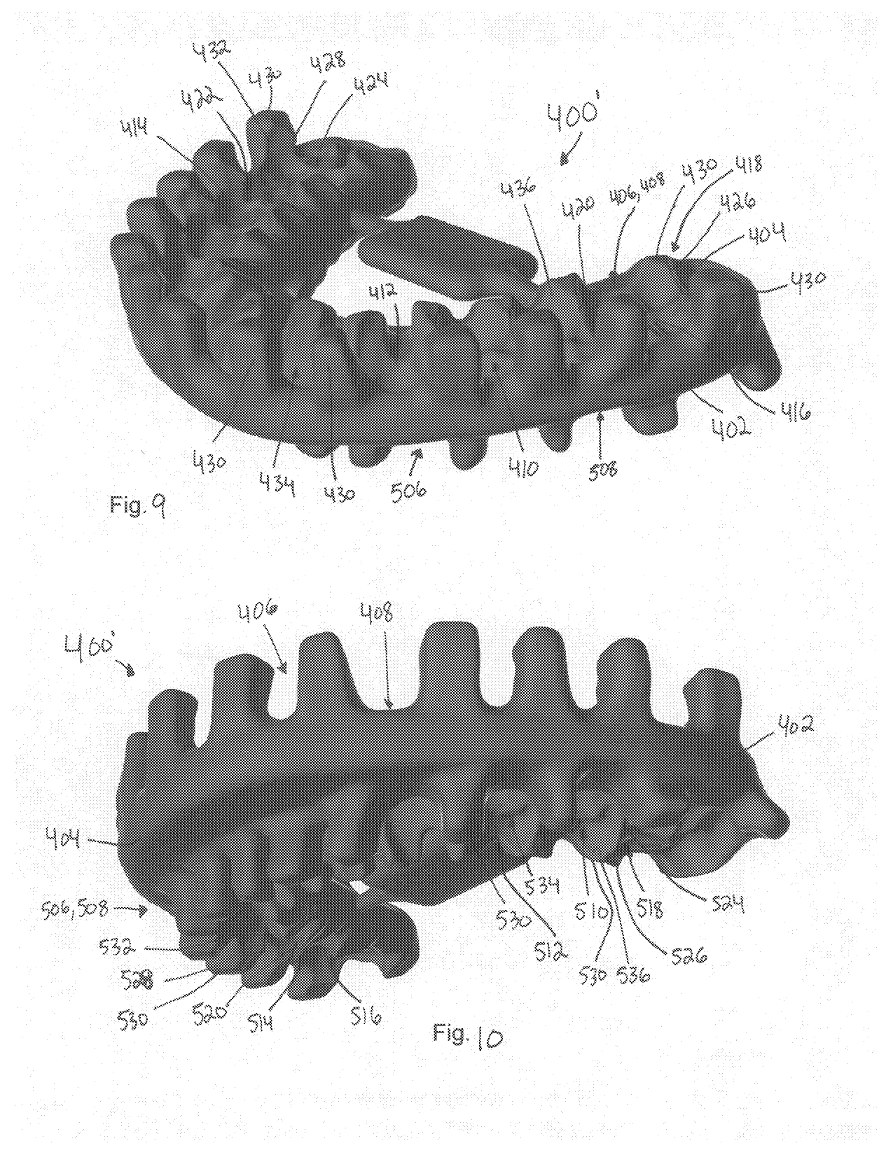

FIG. 8 shows a flowchart for a first example method 800 for performing a bimaxillary surgery. For purposes of simplicity, the method 800 (and, similarly, the method 1300) is shown and described as being executed serially; however, it is to be understood and appreciated that the present disclosure is not limited by the illustrated order as some steps may occur in different orders and/or concurrently with other steps shown and described herein.

At 802, one or more pre-op 3D images of patient’s upper and lower dental arches are captured. For example, the pre-op 3D images may be captured using an imaging and/or scanning device such as, but not limited to, a cone beam computed tomography (CBCT) machine or intraoral scanner.

At 804, the pre-op 3D images are processed using computer-aided design (CAD) software to create a pre-op 3D model of the patient’s upper and lower dental arches. The pre-op 3D model may be manipulated using the CAD software to generate a first planning 3D model that correlates to the patient’s maxilla being in an associated target position and the patient’s mandible being in an associated pre-op position. Using the first planning 3D model, a first bimaxillary splint 3D model may be designed using the CAD software. The first bimaxillary splint 3D model is designed such that the maxillary receptacles 410 of the upper body portion 406 are in an associated target position that corresponds to the target position the patient’s maxilla, while the mandibular receptacles 510 of the lower body portion 506 are in an associated pre-op position that corresponds to the pre-op position of the patient’s mandible. Instructions for creating a physical first bimaxillary splint are generated via computer-aided manufacturing (CAM) software using the first bimaxillary splint 3D model. These instructions may then be used in a 3D printing or other manufacturing process to create the first bimaxillary splint.

Instead of creating a single first bimaxillary splint, multiple first bimaxillary splints may be created. For example, multiple of the same first bimaxillary may be created for redundancy purposes. Multiple first bimaxillary splints may also be created where each first bimaxillary splint provides a different fit with the patient’s maxillary and mandibular dental structures. In particular, at least one standard-fit first bimaxillary splint may be created having a “standard” fit with the patient’s maxillary and mandibular dental structures as computed using the pre-op 3D model and/or the first planning 3D model. At least one tight-fit first bimaxillary splint may be created having an at least slightly tighter fit with the patient’s maxillary and mandibular dental structures than that of the standard-fit first bimaxillary splint. At least one loose-fit first bimaxillary splint may be created having an at least slightly looser fit with the patient’s maxillary and mandibular dental structures than that of the standard-fit first bimaxillary splint. At some point in time before or during the bimaxillary surgery, each of these first bimaxillary splints of differing fits may then be tested/put on the patient to see which provides the most secure and/or desired fit with the patient’s maxillary and mandibular dental structures.

At 806, the pre-op 3D model and/or the first planning 3D model may be manipulated using the CAD software to generate a second planning 3D model that correlates to the patient’s maxilla being in its associated target position and the patient’s mandible being in an associated target position. Using the second planning 3D model, a second bimaxillary splint 3D model may be designed using the CAD software. The second bimaxillary splint 3D model is designed such that the maxillary receptacles 410 of the upper body portion 406 are in the associated target position that corresponds to the target position the patient’s maxilla, while the mandibular receptacles 510 of the lower body portion 506 are in an associated target position that corresponds to the target position of the patient’s mandible. Instructions for creating a physical second bimaxillary splint may be generated via the CAM software using the second bimaxillary splint 3D model. These instructions may then be used in a 3D printing or other manufacturing process to create the second bimaxillary splint.

Instead of creating a single second bimaxillary splint, multiple second bimaxillary splints may be created. For example, multiple of the same second bimaxillary may be created for redundancy purposes. Multiple second bimaxillary splints may also be created where each second bimaxillary splint provides a different fit with the patient’s maxillary and mandibular dental structures. In particular, at least one standard-fit second bimaxillary splint may be created having a “standard” fit with the patient’s maxillary and mandibular dental structures as computed using the pre-op 3D model and/or the second planning 3D model. At least one tight-fit second bimaxillary splint may be created having an at least slightly tighter fit with the patient’s maxillary and mandibular dental structures than that of the standard-fit second bimaxillary splint. At least one loose-fit second bimaxillary splint may be created having an at least slightly looser fit with the patient’s maxillary and mandibular dental structures than that of the standard-fit second bimaxillary splint. At some point in time before or during the bimaxillary surgery, each of these second bimaxillary splints of differing fits may then be tested/put on the patient to see which provides the most secure and/or desired fit with the patient’s maxillary and mandibular dental structures.

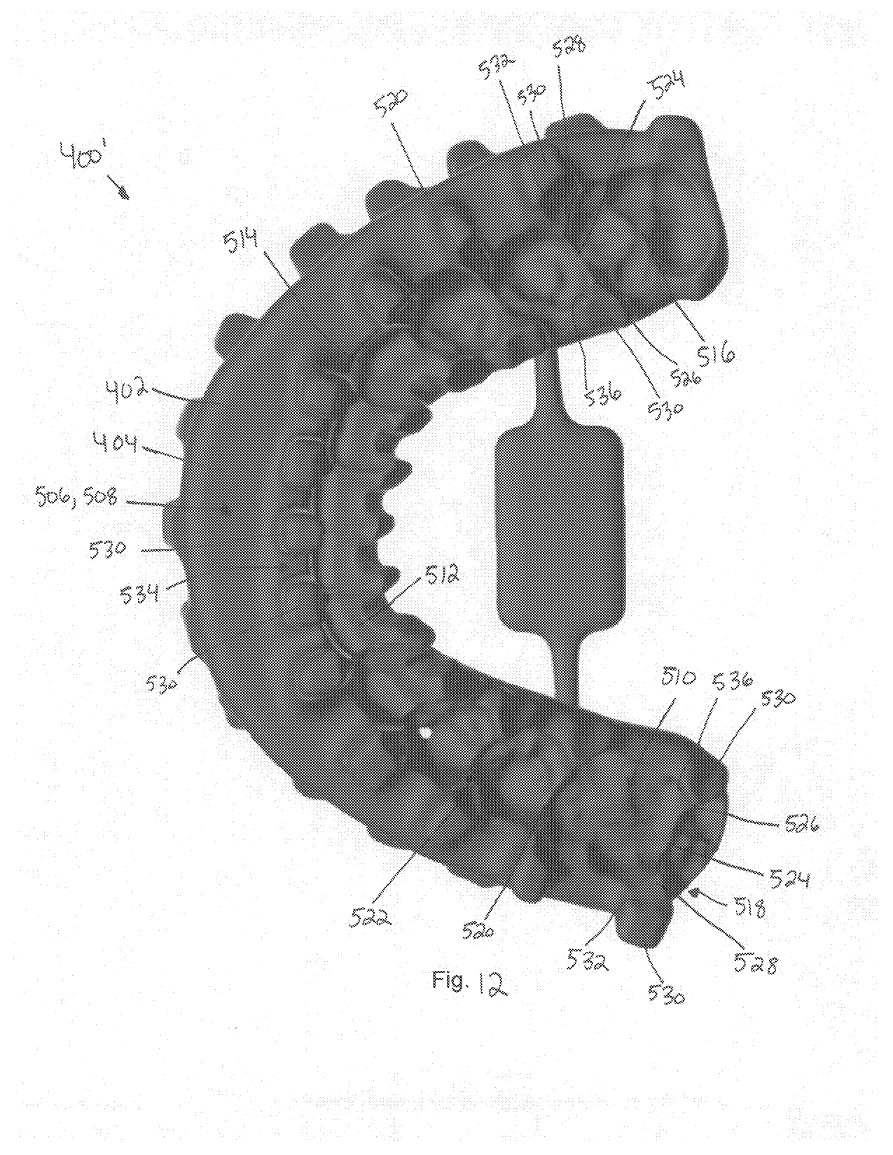

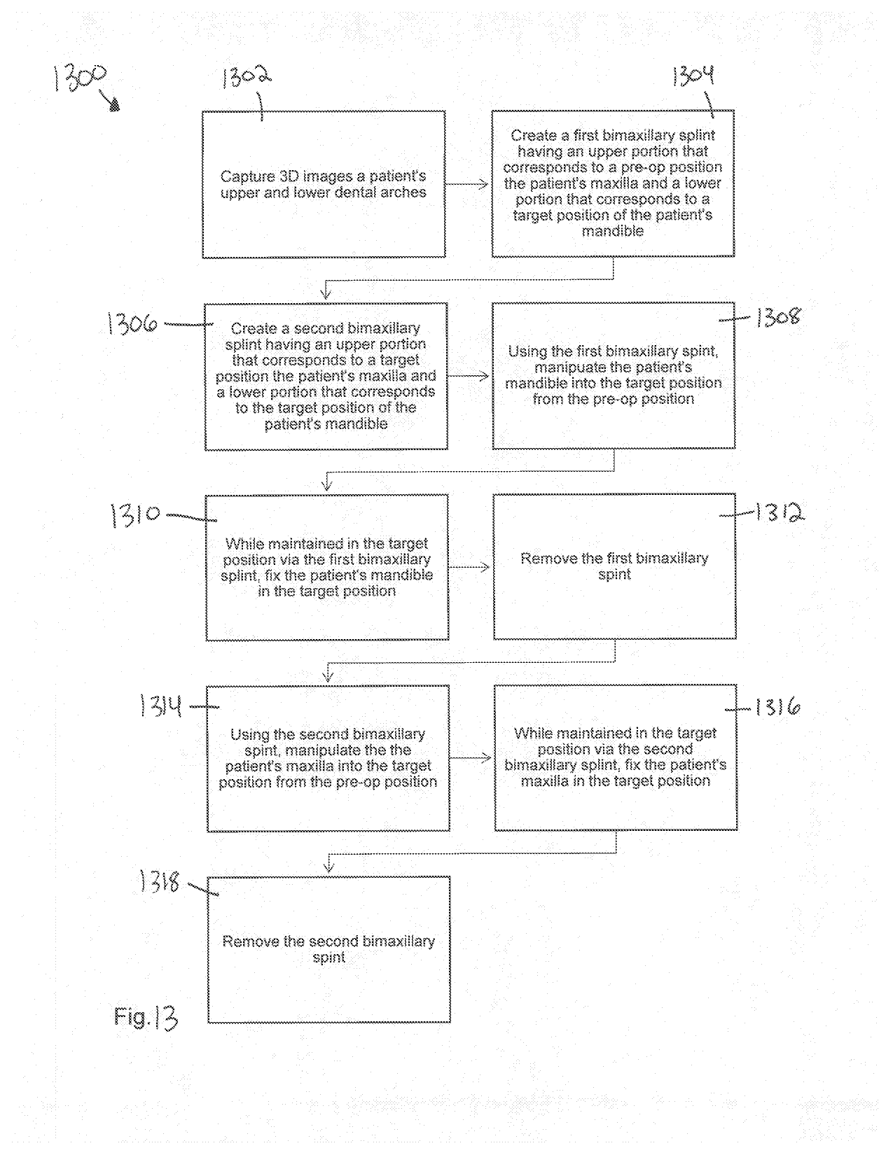

The bimaxillary splint 400 of FIGS. 4-7 illustrates one example of a second bimaxillary splint (i.e., a bimaxillary splint in which the upper and lower body portions 406, 506 and maxillary and mandibular receptacles 410, 510 correspond to the target positions of the patient’s maxilla and mandible). The bimaxillary splint 400ʹ of FIGS. 9-12, on the other hand, illustrate one example of a first bimaxillary splint (i.e., a bimaxillary splint in which the upper body portion 406 and maxillary receptacles 410 correspond to the target position of the patient’s maxilla, and the lower body portion 506 and mandibular receptacle 510 correspond to the pre-op position of the patient’s mandible). The bimaxillary splint 400ʹ of FIGS. 9-12 thus is similar to the bimaxillary splint 400 of FIGS. 4-7 and therefore, structures of FIGS. 9-12 that are the same or similar to those described with reference to FIGS. 4-7 have the same reference numbers, with the exception being reference number “400” having a “prime” mark in FIGS. 9-12. Description of common elements and function similar to those in the previously described bimaxillary splint 400 of FIGS. 4-7 will not be repeated with respect to the bimaxillary splint of FIGS. 9-12, but should instead be considered to be incorporated by reference as appropriate. As can be gleaned from the above, the differences between the bimaxillary splint 400 of FIGS. 4-7 and that of the bimaxillary splint 400ʹ of FIGS. 9-12 are at least in part the result of the bimaxillary splint 400 of FIGS. 4-7 being a “second bimaxillary splint” and the bimaxillary splint 400ʹ of FIGS. 9-12 being a “first bimaxillary splint”.

After being created, the first and second bimaxillary splints may be supplied to a medical professional as a bimaxillary fixation system for use in the bimaxillary surgery, where the geometric configurations of the first and second bimaxillary splints are specific to the patient is or will be undergoing the surgery. The bimaxillary fixation system may optionally include multiple first bimaxillary splints, where there are multiple of the same first bimaxillary splint and/or at least one first bimaxillary that has a different “fit” than at least one other of the first bimaxillary splints, and/or multiple second bimaxillary splints, where there are multiple of the same second bimaxillary splint and/or at least one second bimaxillary that has a different “fit” than at least one other of the second bimaxillary splints.

At 808, the patient’s maxilla is manipulated into the target position from the pre-op position using the first bimaxillary splint. In particular, the medical professional performs an osteotomy (e.g., a Le Fort 1 osteotomy) on the patient’s maxilla. Such an osteotomy allows the medical professional to move the patient's maxilla from its pre-op position. Before or after this osteotomy, the medical professional may join the first bimaxillary splint to the patient’s mandibular dental structures such that the mandibular dental structures are received in the mandibular receptacles 510 of the first bimaxillary splint and the interference fit engagement is formed between the lower body portion 506 and the patient’s mandibular dental structures via the mandibular retention features 518. Once the patient’s mandibular dental structures are engaged to the lower body portion 506 of the first bimaxillary splint and the osteotomy is completed, the medical professional may manipulate (e.g., move) the patient’s maxilla so as to insert the patient’s maxillary dental structures into the maxillary receptacles 410 of the first bimaxillary implant. This osteotomy, manipulation, and insertion results in the patient’s maxilla being moved into its desired target position from its pre-op position. Therefore, the first bimaxillary splint can function as a guide for moving the patient’s maxilla to its target position relative to the patient’s mandible. The interference fit engagement between the upper body portion 406 and the patient’s maxillary dental structures is formed via the maxillary retention features 418 when the patient’s maxillary dental structures are received in the maxillary receptacles 410.

The bimaxillary splints disclosed herein are configured such that when the patient’s maxillary and mandibular dental structures are engaged to the upper and lower body portions, the maxilla and mandible of the patient are prevented (or substantially prevented) from moving relative to one another. This may be in part due to at least the splint body being formed at least partially from an at least partially or substantially rigid material (e.g., an at least partially or substantially rigid polymer). In other words, the rigidity of the splint body may prevent (or substantially prevent) the maxilla and mandible of the patient from moving relative to one another when the patient’s maxillary and mandibular dental structures are engaged to the upper and lower body portions. Therefore, with the first bimaxillary splint engaged to the patient’s maxillary and mandibular dental structures, the patient’s maxilla is maintained (or substantially maintained) in the target position, the patient’s mandible is maintained (or substantially maintained) in a position (e.g., its associated pre-op position), and the patient’s maxilla and mandible are prevented (or substantially prevented) from moving from these positions relative to one another.

At 810, while the patient’s maxilla is maintained in the target position via the first bimaxillary splint, the patient’s maxilla may be fixed in the desired and associated target position via hardware (e.g., plates and/or screws). At 812, the first bimaxillary splint may be disengaged and removed from the patient after the patient’s maxilla is fixed in its target position.

At 814, the patient’s mandible is manipulated into the target position from the pre-op position using the second bimaxillary splint. In particular, the medical professional performs an osteotomy (e.g., a bilateral sagittal osteotomy) on the patient’s mandible. Such an osteotomy allows the medical professional to move the patient's mandible from its pre-op position. Before or after this osteotomy, the medical professional may join the second bimaxillary splint to the patient’s maxillary dental structures such that the maxillary dental structures are received in the maxillary receptacles 410 of the second bimaxillary splint and the interference fit engagement is formed between the upper body portion 406 and the patient’s maxillary dental structures via the maxillary retention features 418. Once the patient’s maxillary dental structures are engaged to the upper body portion 406 of the second bimaxillary splint and the osteotomy is completed, the medical professional may manipulate (e.g., move) the patient’s mandible so as to insert the patient’s mandibular dental structures into the mandibular receptacles 510 of the second bimaxillary implant. This osteotomy, manipulation, and insertion results in the patient’s mandible being moved into its desired target position from its pre-op position. Therefore, the second bimaxillary splint can function as a guide for moving the patient’s mandible to its target position relative to the patient’s maxilla. The interference fit engagement between the lower body portion 506 and the patient’s mandible dental structures is formed via the mandibular retention features 518 when the patient’s mandibular dental structures are received in the mandibular receptacles 510.

With the second bimaxillary splint engaged to the patient’s maxillary and mandibular dental structures, the patient’s maxilla is maintained (or substantially maintained) in its associated target position, the patient’s mandible is maintained (or substantially maintained) in its associated target position, and the patient’s maxilla and mandible are prevented (or substantially prevented) from moving from these positions relative to one another. At 816, while the patient’s mandible is maintained in the target position via the second bimaxillary splint, the patient’s mandible may be fixed in the desired and associated target position via hardware (e.g., plates and/or screws). At 818, the second bimaxillary splint may be disengaged and removed from the patient after the patient’s mandible is fixed in its target position.

It should be appreciated from the above that a bimaxillary splint designed in accordance with the teachings herein may be quickly and easily engaged to a patient and utilized to achieve the desired positioning and/or fixation of the patient’s maxilla and/or mandible without the use of fasteners (e.g., metallics fasteners), wire loops, dental cement bonding, and/or arch bars (e.g., metallic arch bars). Therefore, removing the bimaxillary splints from the patient is also relatively quick, easy, and cost effective since no fasteners, wire loops, dental cement, and/or arch bars have to be removed from the patient during and/or after the bimaxillary surgery.

Although the method 800 described above includes manipulating the patient’s maxilla into its target position before the mandible is manipulated into its target position, a system and first and second bimaxillary splints of the system may instead be adapted to be utilized in a bimaxillary surgery method (e.g., method 1300 of FIG. 13) in which a patient’s mandible is manipulated into its target position prior to the manipulation of the patient’s maxilla into its target position. The method 1300 of FIG. 13 thus is substantially similar to that of the method 800 of FIG. 8, with the order in which the patient’s maxilla and mandible are manipulated into and fixed in their associated target positions being reversed. Therefore, for simplicity and brevity, the method 1300 of FIG. 13 has been briefly summarized in the following, where a detailed understanding of each step of the method 1300 of FIG. 13 can be gleaned from its associated step in the method 800 of FIG. 8. The method 1300 of FIG. 13 includes capturing 3D images of a patient’s upper and lower dental arches (1302), creating at least one first bimaxillary splint having an upper body portion 406 that corresponds to a pre-op position of the patient’s maxilla and a lower body portion 506 that corresponds to a target position of the patient’s mandible (1304), creating at least one second bimaxillary splint having an upper body portion 406 that corresponds to a target position of the patient’s maxilla and a lower body portion 506 that corresponds to the target position of the patient’s mandible (1306), manipulating the patient’s mandible into its associated target position from its associated pre-op position using the first bimaxillary splint (1308), fixing the patient’s mandible in the target position while maintaining the patient’s mandible in the target position via the first bimaxillary splint (1310), removing the first bimaxillary splint (1312), manipulating the patient’s maxilla into its associated target position from its associated pre-op position using the second bimaxillary splint (1314), fixing the patient’s maxilla in the target position while maintaining the patient’s maxilla in the target position via the second bimaxillary splint (1310), and removing the second bimaxillary splint (1312).

Although the bimaxillary splints 400, 400ʹ of the present disclosure have been utilized in a bimaxillary surgery, a bimaxillary splint 400/400ʹ designed in accordance with the present disclosure may be utilized for any other purpose and/or in any other procedure in which intermaxillary fixation is desired and/or beneficial. In other words, a bimaxillary splint 400/400ʹ designed in accordance with the present disclosure 400/400ʹ may be adapted to be utilized in any procedure in which a relative fixation of a patient’s maxilla and mandible is desired and/or beneficial, where the specific geometry and configuration of the bimaxillary splint 400/400ʹ is patient and/or procedure specific.

It is also contemplated that any of the bimaxillary splints 400/400’ of the present disclosure may also or instead be configured to at least partially engage with an anatomic undercut of the mandible and/or the maxilla, potentially in combination with occlusal retention. This undercut-engagement of the bimaxillary splints 400/400’ would be similar, for example, to the manner in which a lingual denture is retained by engaging a posterior lingual undercut of the mandible.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials; however, the chosen materials should be biocompatible for many applications. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. The term “substantially” is used herein to indicate a quality that is largely, but not necessarily wholly, that which is specified--a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Other aspects, objects, and advantages may be obtained from a study of the drawings, the disclosure, and the appended claims.

I claim:

1. A bimaxillary splint for fixing each of the maxilla and mandible of a patient in a selected position, the bimaxillary splint comprising:
a substantially U-shaped splint body having opposing upper and lower body portions, the upper body portion having a plurality of maxillary receptacles for receiving associated maxillary dental structures of the patient, the lower body portion having a plurality of mandibular receptacles for receiving associated mandibular dental structures of the patient;
a plurality of maxillary retention features on the upper body portion in and/or adjacent to the maxillary receptacles, the maxillary retention features being configured to engage the maxillary dental structures and/or interproximal spaces between adjacent maxillary dental structures to form an interference fit engagement between the upper body portion and the patient’s maxillary dental structures; and
a plurality of mandibular retention features on the lower body portion in and/or adjacent to the mandibular receptacles, the mandibular retention features being configured to engage the mandibular dental structures and/or interproximal spaces between adjacent mandibular dental structures to form an interference fit engagement between the lower body portion and the patient’s mandibular dental structures.

2. The bimaxillary splint of claim 1, wherein the bimaxillary splint is configured such that when the patient’s maxillary and mandibular dental structures are engaged to the upper and lower body portions, the maxilla and mandible of the patient are substantially prevented from moving relative to one another.

3. The bimaxillary splint of claim 2, wherein at least the splint body is formed from a rigid material, the rigidity of the splint body substantially preventing the maxilla and mandible of the patient from moving relative to one another when the patient’s maxillary and mandibular dental structures are engaged to the upper and lower body portions.

4. The bimaxillary splint of claim 1, wherein the splint body is formed as a one-piece, monolithic construction.

5. The bimaxillary splint of claim 1, wherein the splint body, the maxillary retention features, and the mandibular retention features are integrally formed together as a one-piece, monolithic construction.

6. The bimaxillary splint of claim 1, wherein the maxillary retention features comprise maxillary retention projections that each extend from an inner surface of the upper body portion, each maxillary retention projection being in line with and/or defining a maxillary division between two serially adjacent maxillary receptacles such that each maxillary retention projection is configured to extend into an associated interproximal embrasure of the patient’s maxillary dental structures.

7. The bimaxillary splint of claim 6, wherein each maxillary retention structure has at least one of:
an occlusal/incisal projection portion configured to extend upward toward and/or into an occlusal/incisal embrasure portion of an associated interproximal embrasure;
a lingual projection portion configured to extend facially toward and/or into a lingual embrasure portion of an associated interproximal embrasure; and
a facial projection portion configured to extend lingually toward and/or into a facial embrasure portion of an associated interproximal embrasure.

8. The bimaxillary splint of claim 6, wherein the splint body comprises a base having an upper base portion in which the maxillary receptacles are formed and a lower base portion in which the mandibular receptacles are formed, the splint body further comprising a plurality of maxillary retention tabs that extend substantially vertically upward from the upper base portion, each maxillary retention tab having an inner surface from which at least a portion of an associated maxillary retention projection extends in a lingual or facial direction.

9. The bimaxillary splint of claim 8, wherein the maxillary retention tabs are spaced from one another along a substantially U-shaped extension of the base.

10. The bimaxillary splint of claim 1, wherein the mandibular retention features comprise mandibular retention projections that each extend from an inner surface of the lower body portion, each mandibular retention projection being in line with and/or defining a mandibular division between two serially adjacent mandibular receptacles such that each mandibular retention projection is configured to extend into an associated interproximal embrasure of the patient’s mandibular dental structures.

11. The bimaxillary splint of claim 10, wherein each mandibular retention structure has at least one of:
an occlusal/incisal projection portion configured to extend downward toward and/or into an occlusal/incisal embrasure portion of an associated interproximal embrasure;
a lingual projection portion configured to extend facially toward and/or into a lingual embrasure portion of an associated interproximal embrasure; and
a facial projection portion configured to extend lingually toward and/or into a facial embrasure portion of an associated interproximal embrasure.

12. The bimaxillary splint of claim 10, wherein the splint body comprises a base having an upper base portion in which the maxillary receptacles are formed and a lower base portion in which the mandibular receptacles are formed, the splint body further comprising a plurality of mandibular retention tabs that extend substantially vertically downward from the lower base portion, each mandibular retention tab having an inner surface from which at least a portion of an associated mandibular retention projection extends in a lingual or facial direction.

13. The bimaxillary splint of claim 12, wherein the mandibular retention tabs are spaced from one another along a substantially U-shaped extension of the base.

14. The bimaxillary splint of claim 1, wherein each maxillary receptacle is defined at least by an inner lingual-side surface of the upper body portion and an inner facial-side surface of the upper body portion, the maxillary retention features comprising the inner lingual-side surfaces and the inner facial-side surfaces, a distance between the inner lingual-side surface and the inner facial-side surface at each maxillary receptacle being configured such that the inner lingual-side surface and the inner facial-side surface engage the maxillary dental structures on opposing sides of the maxillary dental structures to such a degree that the interference fit engagement is formed between the upper body portion and the patient’s maxillary dental structures.

15. The bimaxillary splint of claim 1, wherein each mandibular receptacle is defined at least by an inner lingual-side surface of the lower body portion and an inner facial-side surface of the lower body portion, the mandibular retention features comprising the inner lingual-side surface and the inner facial-side surface, a distance between the inner lingual-side surface and the inner facial-side surface at each mandibular receptacle being configured such that the inner lingual-side surface and the inner facial side surface engage the mandibular dental structures on opposing sides of the mandibular dental structures to such a degree that the interference fit engagement is formed between the lower body portion and the patient’s mandibular dental structures.

16. The bimaxillary splint of claim 1, wherein a geometric configuration of the bimaxillary splint is patient specific.

17. A bimaxillary fixation system for use in a bimaxillary surgery, the bimaxillary fixation system comprising:
a plurality of the bimaxillary splint of claim 1, the plurality of bimaxillary splints comprising first and second bimaxillary splints,
the first bimaxillary splint being configured such that one of the maxillary and mandibular receptacles are in an associated pre-op position while the other of the maxillary and mandibular receptacles are in an associated target position,
the second bimaxillary splint being configured such that the maxillary and mandibular receptacles are in their associated target positions.

18. The bimaxillary fixation system of claim 17, wherein the target position of the maxillary receptacles corresponds to a target position of a maxilla of the patient, the target position of the maxilla being different than that of a pre-op position of the maxilla, and
wherein the target position of the mandibular receptacles corresponds to a target position of a mandible of the patient, the target position of the mandible being different than that of a pre-op position of the mandible.

19. The bimaxillary fixation system of claim 18, wherein the pre-op position of the maxillary receptacles corresponds to the pre-op position of the maxilla, and
wherein the pre-op position of the mandibular receptacles corresponds to the pre-op position of the mandible.

20. A method for performing a bimaxillary surgery, comprising:
providing the bimaxillary fixation system of claim 17;
through use of the first bimaxillary splint, manipulating a first one of the maxilla and mandible into an associated target position from an associated pre-op position;
with the first one of the maxilla and mandible maintained in its associated target position via the first bimaxillary splint, fixing the first one of the maxilla and mandible in its associated target position;
after fixing the first one of the maxilla and mandible in its associated target position, removing the first bimaxillary splint;
through use of the second bimaxillary splint, manipulating a second one of the maxilla and mandible into an associated target position from its associated pre-op position;
with the maxilla and mandible maintained in their associated target positions via the second bimaxillary splint, fixing the second one of the maxilla and mandible in its associated target position; and
after fixing the second one of the maxilla and mandible in its associated target position, removing the second bimaxillary splint.
